(19) United States
(12) Patent Application Publication
(10) Pub. No.: US 2008/0166792 A1
(43) Pub. Date: Jul. 10, 2008

(54) DETECTION OF ANALYTES IN MATERIALS LIQUIDS USING CAPILLARY COLORIMETRIC DETECTION

(76) Inventors: Amir J. Attar, Raleigh, NC (US); Dan Edward Stark, Raleigh, NC (US)

Correspondence Address: INTELLECTUAL PROPERTY / TECHNOLOGY LAW, PO BOX 14329, RESEARCH TRIANGLE PARK, NC, 27709, US

(21) Appl. No.: 11/620,233

(22) Filed: Jan. 5, 2007

(51) Int. Cl. C12M 1/00; G01N 21/00

(52) U.S. Cl. 435/287.2; 422/58

(57) ABSTRACT

Systems and methods for the rapid and reliable detection of analytes in liquid solutions such as water, drinking fluids, extracts of solids such as foods, soils, industrial fluids such as oils, cooling water, fuels, solutions of drugs or chemicals, etc. The systems preferably include an inexpensive and disposable capillary containing a dry chemical system of detection that reacts chromogenically or in other manner to indicate the presence of the analyte.

### FIELD OF THE INVENTION

The present invention relates to the use of capillaries as containers of media that react with ingredients that enter the capillaries by diffusion or by capillary action to form a measurable change such as a change in color, in which the color change is characteristic of a component of the fluid or of a group of materials with common characteristics. These devices, referred to as Capillary Detectors, (CD), can be used individually or as components of a system adapted for detecting one or more materials. Examples of the use of systems of CDs include detection of analytes in food, or contaminants or bacteria in water.

### BACKGROUND OF THE INVENTION

The need to determine quickly if a liquid contains specified materials is faced frequently in many fields. Examples include the detection of analytes or adulterants in drinking water and other fluids, the need to detect biological materials such as proteins or ketones in urine, the need to detect biohazards in water and other fluids, the need to determine if specific fluids contain materials that can affect their properties, such as detecting chromates or chlorides in industrial fluids, and even detecting hydrogen peroxide or acetone in fluids carried by passengers into aircraft. Although many analytical methods are available to address these problems, the available methods are expensive, lengthy, require complex instrumentation which cannot be easily handled by laymen or cannot be adopted to use in the field.

Adulteration of water by terrorists using hazardous biological materials or other analytes, has become a real threat and its implementation a realistic possibility. Threats have been made to poison unsuspecting random people around the world. As such, we can no longer take for granted that the food and/or water we consume are free of artificial poison(s).

Recent discoveries of liquid precursors to explosives in the hands of terrorists seeking to board aircraft has forced yet again another shift in the security procedures carried out in airports, and resulted in profound changes in search procedures used, as well as in the types of materials that passengers are allowed to bring on board.

A very large amount of money and other resources have been invested in developing methods for quick analysis of liquids, however, most of the available methods require very skilled labor, expensive instruments, access or proximity to well-equipped laboratory facilities, etc. Needless to say, the results of many of the available methods are not obtained in real time and thus cannot address contemporaneous needs where having an instantaneous result on site is critical to making a correct informed decision.

The objective of this invention is to describe a general, very low-cost and simple method for determining in real time if specific components are present in a liquid. The material to be detected, the analyte, may be inorganic, organic, biological or even a live microorganism. Examples will be described hereinafter where low cost CDs were constructed and used to determine such materials in solution using the CD.

A useful method for detecting an analyte in solution should be quick, reliable, easily applied, and the results unambiguously understood. In addition, it should be designed so that false negative and false positive errors are eliminated. From a practical point of view, the method and hardware should be relatively low cost, stable and compact, so that they can be widely disseminated to a wide range of users, both private and professional, and be readily available for use anywhere.

Towards that end, the present invention relates to a methodology and systems to rapidly and reliably determine if a fluid such as water contains acutely dangerous amounts of specific analytes, chemicals, biochemicals, biohazards etc., with no additional instrumentation besides the simple hardware supplied to the users.

### SUMMARY OF THE INVENTION

It is therefore an object of the present invention to describe a generic apparatus for the detection of analytes in liquids.

A further object of the present invention is to describe a generic apparatus for the detection of analytes in liquids that is inexpensive and disposable.

In one implementation, the present invention relates to a calorimetric detection device for sensing the presence and/or the identity of at least one analyte in a liquid sample. Such detection device comprises:
a capillary made of a transparent material, such as glass or plastic, where one end of the capillary is open or easily openable to permit the introduction of the liquid sample by capillary action,
a porous powder or porous monolithic solid support;
a porous plug at the open or openable end of the capillary to retain the powder,
a color-forming chemical that changes color in response to contact or reaction with at least one said analyte, wherein the color-forming chemical is disposed on or in said support and is visible through the transparent capillary walls,

Another implementation of the present invention relates to a method of sensing the presence and identity of at least one analyte in a liquid sample, said method comprising:
a capillary made of a transparent material, such as glass or plastic, where one end of the capillary is open or easily openable to permit the introduction of the liquid sample by capillary action,
multiple layers of porous powder or porous monolithic solids supports, placed consecutively in the capillary and separated by small porous plugs;
a porous plug at the open or openable end of the capillary to retain the powder,
a color-forming chemical that changes color when interacting with at least one said analyte or with a secondary reaction product of said analyte, wherein the color-forming chemical is disposed on or in one of said supports,
disposing chemicals on or in at least a portion of the support layer to condition the solution so it will form a color change in response to exposure to at least one said analyte;
allowing the liquid sample to pass through said opening and contact said color-forming chemical, causing the same to change color; and
evaluating the resulting color of said color-forming chemical through the transparent walls of the capillary to determine the identity and/or concentration of said at least one analyte,
wherein said liquid sample comprises a sample selected from the group consisting of water, liquid food, an extract of solid food, soil, extract of the content of the stomach, extract of feces, urine, ground water, waste water, wash water as well as industrial water, wash water of foods or vegetables, when looking for bacteria or viruses, bodily fluids such as urine, blood, plasma etc.

Another implementation of the present invention relates to a method of sensing the presence and identity of at least one analyte in a liquid sample, said method comprising:
a capillary made of a transparent material such as glass or plastic where one end of the capillary is open or easily openable to permit the introduction of the liquid sample by capillary action,
multiple layers of porous powder or porous monolithic solids supports placed consecutively in the capillary and separated by small porous plugs;
a porous plug at the open or openable end of the capillary to retain the powder,
a color-forming chemical that changes color when interacting with said at least one gaseous reaction product of said analyte wherein the color-forming chemical is disposed on or in one of said support layer,
disposing chemicals on or in some of the support layer to condition the solution so it will form a color change in response to exposure to said at least one analyte;
disposing chemicals on or in at least a portion of the support layer to react with said analyte or with one of its reaction products in the solution to form a gaseous reaction product that subsequently reacts and form a color change in response to said at least one analyte;
allowing the liquid sample to pass through said opening and said plug so that the liquid sample will contact some of the reagents placed on some of the support materials to form the gas which subsequently reacts and forms a color change, and
evaluating the resulting color of said color-forming chemical through the transparent walls of the capillary to determine the identity and/or concentration of said at least one analyte,
wherein said liquid sample comprises a sample selected from the group consisting of water, liquid food an extract of solid food, soil etc, extract of the content of the stomach, extract of feces, urine, ground water, waste water, wash water as well as industrial water, wash water of foods or vegetables, when looking for bacteria or viruses, bodily fluids such as urine, blood, plasma etc.

In another implementation of the present invention relates to a method of sensing the presence and identity of at least one analyte in a liquid sample, wherein each layer of support material may consist of a mixture of several powders bearing different reagents to facilitate parallel or second order reactions to take place while maintaining the stability of the layer and the chemicals it supports.

Other aspects, features and embodiments of the invention will be more fully apparent from the ensuing disclosure and appended claims.

### BRIEF DESCRIPTION OF THE DRAWINGS

FIG. 1 is a cross-sectional view of the simplified embodiment of the analyte detection capillary of the present invention.

FIG. 2 is a cross-sectional view of the simplified embodiment of the analyte detection capillary of FIG. 1 using two consecutive layers of materials to effect the desired final color change.

FIG. 3 is a cross-sectional view of another embodiment of the analyte detection capillary of the present invention where the sequence of reactions in the conditioning layers produces a gas which is detected in a subsequent chromophoric layer.

FIG. 4 is a cross-sectional view of the embodiment of the analyte detection capillary of FIG. 3 where gradations were added to allow semi-quantification of the amount of gas produced and thus the analyte concentration in the solution.

FIG. 5 is a cross-sectional view of yet another embodiment of the analyte detection capillary of the present invention where a mixture of chromogen-laden solid particles are premixed and placed in a single layer.

FIG. 6 is a cross-sectional view of yet another embodiment of the analyte detection capillary of the present invention where several layers of chromogen-laden solid particles are used to allow the detection of several analytes in a single sample and where the sample is allowed in from one side only.

FIG. 7 is a cross-sectional view of yet another embodiment of the analyte detection capillary of the present invention where several layers of chromogen-laden solid particles are used to allow the detection of several analytes in a single sample and where the sample is allowed in from both sides of the capillary.

FIG. 8 illustrates top and bottom views of an embodiment of the analyte detection capillary of the present invention that can be analyzed in semi-quantitative tests using an electronic reader.

FIG. 9 illustrates top and bottom views of an embodiment of the analyte detection capillary of the present invention that can be used in conjunction with an electronic reader to trigger the opening or closing of valves, to turn pumps on or off, etc.

FIG. 10 shows an embodiment of the analyte detection capillary of the present invention that uses a porous oleophobic material at the entrance to the capillary to prevent the admission of oil droplets into the capillary.

### DETAILED DESCRIPTION OF THE INVENTION, AND PREFERRED EMBODIMENTS THEREOF

The present invention relates to an apparatus and method of using dry chemical techniques to detect analytes in fluids such as water, food extracts, fuels, industrial liquids and many other fluids. The presence of the desired analyte is indicated when the color of the support layer carrying the chromophore changes from one color to another. Other indicators, such as a change in fluorescence, may be used in special cases. As will be discussed herein, the present invention introduces several innovations to analyte detection including, but not limited to, the use of capillaries both as the sampling means for liquids as well as the containers for dry chemical in various forms to rapidly and simultaneously detect specific or classes of analytes, while eliminating interferences from secondary materials such as food ingredients or other components of the solution carrying matrix, reducing the sample preparation work needed, simplifying the result interpretation and qualitative and quantitative identification of the specific analyte detected.

The detection capillary of the present invention can be used to test for analyte contained in body fluids, e.g. for forensic purposes, environmental samples, industrial water, waster water, fluids from waste dumps, fluids from chemical processing facilities, etc.

It is noted that the porous entrance plug into the capillary may be made from different materials including porous oleophobic materials that deny droplets of oil entry into the capillary. This eliminates the need to use time consuming sample-preparation procedures to remove oils, particles, or in some cases to extract or concentrate the sample and thereby provide for a more rapid test.

As defined herein, “sample” is used generically and includes, without limitation, ingestible substances such as drinking water, ground water, all liquid and/or extractable solid foods, soil, biological samples, etc., process water, such as water from cooling tower, solutions of pharmaceuticals and solutions of intermediates used in making drugs, chemicals etc., bodily fluids such as urine and blood, substances used to cook e.g. oils, substances used to flavor liquid and/or solid foods (e.g., spices and other powders), and the materials in which the solid foods are cooked or washed. It is to be understood that these specific references are not meant to be limiting in any way, but rather to describe the broad scope of applications of this technique.

In most embodiments, the present invention utilizes no electronic instruments, sensors computers, power sources and other ancillary resources and it does not require calibration. In other words, the presence of analyte is detected calorimetrically using visual methods.

A quality assurance procedure (QA) may be built into the test procedure of all the embodiments as a method to reduce the number of false positive determinations and essentially eliminate the number of false negative determinations. This is done by using a second capillary along with the analytical one and using it to test a known liquid.

In another embodiment of the present invention, the use of calorimetric instruments is required to determine the analyte concentration based on the length of the color stain formed in the analyte detection.

In another embodiment of the present invention, a calorimetric instrument is used to trigger a secondary activity such as turning on a pump, opening a pneumatic valve, etc.

Although some of the elements of the chemistries described herein are known in the art, the methodology of their use, as well as the apparatuses in which they are incorporated, are new. The apparatus described herein eliminates or reduces interferences from various components of the carrying liquid matrix. Special verification tests were conducted to validate the applicability of the methodology and apparatus of the present invention to a wide range of various solutions including drinks, foods, cooking materials etc. The apparatus is a flexible system that can be used to detect one analyte or to determine systematically if any analyte, selected from a group of analytes, is present in the carrying solution. Furthermore, the results are unambiguously understood and as such, the methodology may be practiced by a wide range of users including lay people with only minimal training or knowledge of chemistry.

The methodology of the present invention, as described herein, is specifically directed to seven groups of analytes, but may be easily expanded to include other groups of analytes as well. The seven groups of analytes discussed herein are: anionic analytes, cationic analytes, analytes that can be induced to release a characteristic gas, water soluble organic analytes, water insoluble organic analytes in organic media or in suspension, liquid samples containing biological materials such as proteins, ketones, glycerides etc., and liquids containing live bacteria or viruses, etc.

The sensitivities of the methods of the present invention may be adjusted by changing the loadings of the reagents or the chromophores used in the various layers in the capillary detector.

The second technology described herein, i.e., screening tests, is used to detect or respond to multiple materials or analytes in a single test. Preferably, the apparatus of the present invention includes a chromophore, which changes to a unique color upon exposure to a unique analyte. It is also contemplated herein that the present methodology and process may include a screening reagent that changes to the same or to different colors upon contact with different materials or analytes.

For example, it is well known in the art that many metal cations react with the sulfide ion and form a colored compound. This is used in a generic test for the presence of metal cations. The color formed can sometimes provide presumptive identification of the specific metallic cation. These chemistries have been used in numerous qualitative analytical methodologies, however, heretofore have not been implemented in a dry chemical format for screening purposes.

The third technology described herein, i.e., validation of a positive result, is performed using dry chemical tabs including the screening reagent wherein the screening reagent responds specifically to the presence of a specific target analyte by changing colors. Embodiments of these chemistries have been described previously by Fiegl et. al. (“Spot Tests in Inorganic Analysis,” Elsevier Pub. Comp., Amsterdam, (1972)), Feigl F. (“Spot Tests in Organic Analysis,” Elsevier Pub. Comp., Amsterdam, (1956)), Junreis, E. (“Spot Tests Analysis,” John Wiley and Sons, New York, (1985)), and Badcock, N. R. (“Detection of analyteing by Substances other than Drugs: A Neglected Art”, Am. Clin. Biochem., 37, 146-157, (2000)) using spot test plates or impregnated papers. Such tests utilize liquid reagents and often require that the reagents be freshly prepared right before use and/or require special pretreatment conditions or heating. In contrast, the validation capillary detectors of the present invention eliminate the need for heating as well as the need to freshly prepare detection reagents.

Another novel technology is described herein, i.e., the option to include quality assurance (QA), to ensure that the detection capillary has operated properly, the chromophore is still effectively viable, and that no false negative or false positive occurred during the testing for analytes. The QA methodology has been designed to be fast and simple, so that the accurate testing of the samples for analytes can be completed in a relatively very short time. The QA process includes the addition of a second detection capillary and a known amount of analyte-containing pocket to the second capillary to validate the sensitivity of the screening reagent and thus effectively verify the validation process.

One embodiment of the present invention corresponds to a calorimetric detection capillary, in which the detection capillary includes a support layer having an amount of a chromophoric material in or on a porous support material. The chromophore may be dispersed on or in such support layer as micro- or nanoparticles, embedded or impregnated in a thin polymeric film deposited on the surface of the solid support. The chromophoric material is selected so that it reacts with the target analyte(s) to form a visible color change. The color change may be unique to the target analyte or to a group of analytes.

The chromophores of the present invention may include a species selected from the group consisting of molybdates, phosphomolybdates, tungstates, phosphotungstates, iron salts such as sulfates, metallic sulfides such as zinc, calcium, barium, aluminum or strontium sulfides, organic materials such as 8-hydroxy-quinoline and its derivatives, 1-(2-pyidylazo)-2-napthol (PAN) and related compounds that include azo derivatives of heterocyclic compounds, rubeanic acid, diethyldithiocarbamate, dithizone, zincon, diphenylcarbazone, diphenylcarbazide (DPC) rhodizonic acid and its salts, titan yellow, cadion, functionalized diazonium salts including arsenic and phosphonic diazonium salts, triphenylmethane and xanthenes and other materials used in the spectrometric, fluorometric or colorimetric determination of species. Preferably, the chromophore includes a mixture of iron (II) and iron (III) sulfate compounds. The chromophoric mixture optionally includes acids, bases, preservatives, reactants, oxidizing agents, reducing agents, chelating agents, buffers, stabilizers, etc. The chromophore used herein for illustration purposes is a mixture of iron sulfates to detect cyanides, azides and sulfides.

The support layer within the detection capillary may be as simple as paper pulp or shredded blotter paper or as sophisticated as microparticles of activated silica or alumina on a polymeric support wherein the chromophore is on or in the microparticulate material. Other support layers include, but are not limited to polymeric or glass beads, porous membranes, layered fibers and metallic films

The support layer may be chemically inert or it may be capable of assisting the reaction in some way. For example, the support layer may be acidic or basic. Other materials such as buffers, stabilizers or chelating agents may be incorporated within the chromophoric layer to facilitate the chromophoric reaction, prevent interferences, extend the shelf life of the chromophore, and increase its photostability. Importantly, the support layer must ensure maintenance of the chromophores on or in the support layer, must be physically and chemically capable of withstanding exposure to a variety of liquids, and must be non-reactive towards the chromophore and other ingredients in the chromophoric formulation. Optionally, the support layer may be liquid permeable.

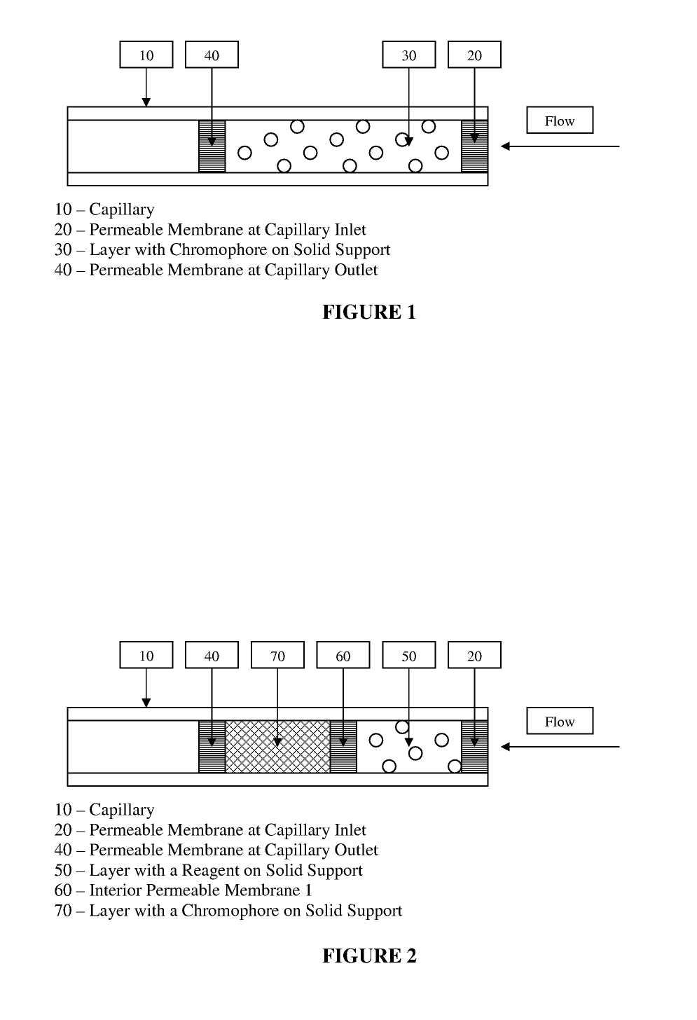

Referring to FIG. 1 the cross-sectional view of an embodiment of the simplified detection capillary 10 is illustrated. The liquid sample is sucked in via capillary forces through the porous plug 20 and onto the aforementioned support layer 30, which includes the chromophore thereon or therein. This support may optionally include also other materials such as soluble buffers or reactants that may remove specific interferents from the test solution. Once the liquid reaches the chromophore in 30, the analyte reacts with it and forms a color change that indicates positive detection.

Written information identifying and/or quantifying the analyte and any other useful information may be printed on a paper onto which the capillary is glued, to assist in interpreting the results and comparing colors.

In another embodiment of the present invention is shown in FIG. 2, where the liquid is again sucked into the capillary through the porous plug 20 and through a conditioning layer 50 and then through the porous plug 60 into the chromogenic layer 70. Layer 50 contains a reagent on a solid porous support which conditions the solution or the analyte to react with the chromophore in 70.

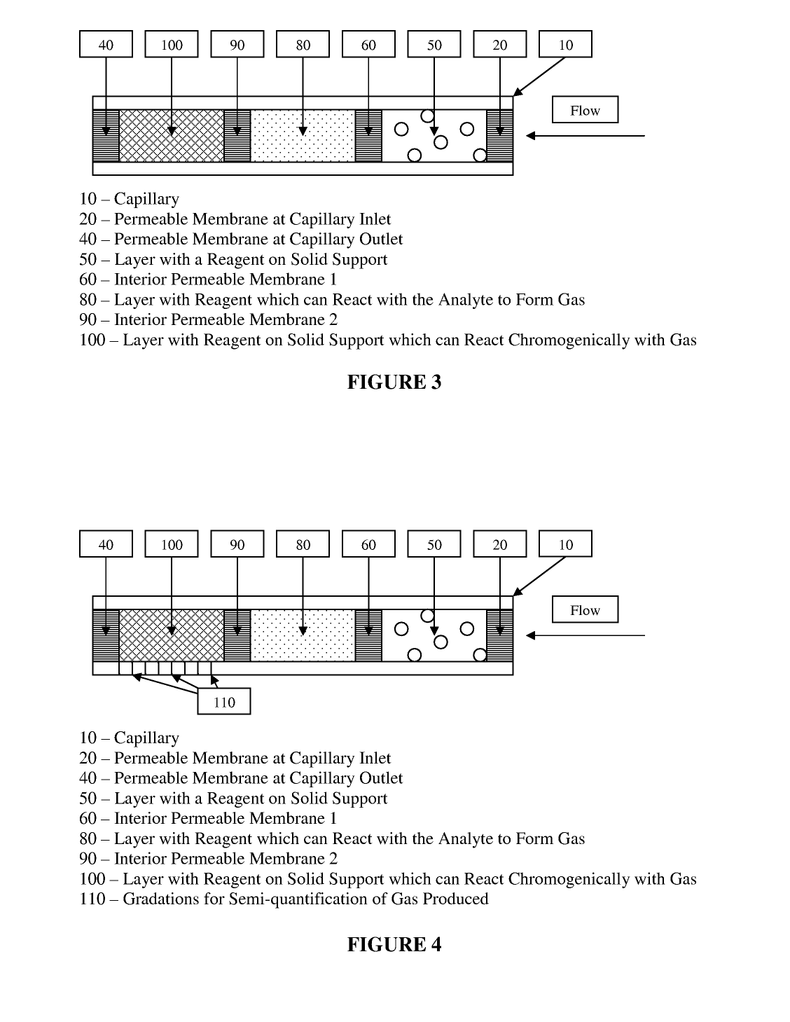

FIG. 3 shows another embodiment of the capillary detector of this invention where the solution of the analyte enters the capillary through the porous plug 20, picks a reagent from the support 50 and carries such extracted reagent with it into the layer 80 through the porous plug 60, where the analyte and two reagents react to form a reactive gas. The reactive gas permeates through the porous plug 90 and then permeates through porous plug 90 into the chromophore adsorbed on the solid support 100 to react and form color.

FIG. 4 shows the cross-section of the same embodiment of the detection capillary of this invention as described in FIG. 3 except that a scale 110 was added near the chromophoric zone 100 to permit the user to estimate the concentration of the analyte based on the length of the color stain formed along the scale. The scale can be etched onto the capillary or printed on a paper attached to the back of the capillary.

In yet another embodiment of the present invention, the chromophore is deposited on or in one solid support and a second reagent is deposited on a second solid support and the two solid supports are mixed together at a controlled ratio to form a single layer that can react with the analyte to form color. The reagent in the second support layer can help filter out solids and other interfering materials, to host conditioning materials such as pH buffers, materials that remove selectively interfering materials, etc. FIG. 5 shows the simplest implementation of these design where the liquid enters the capillary 10 through the porous plug 20 into the mixed layer 120 where the mixture of the two solids carrying the chromophore and the reagent is present. When the liquid solution contains the analyte, color will be formed in layer 120.

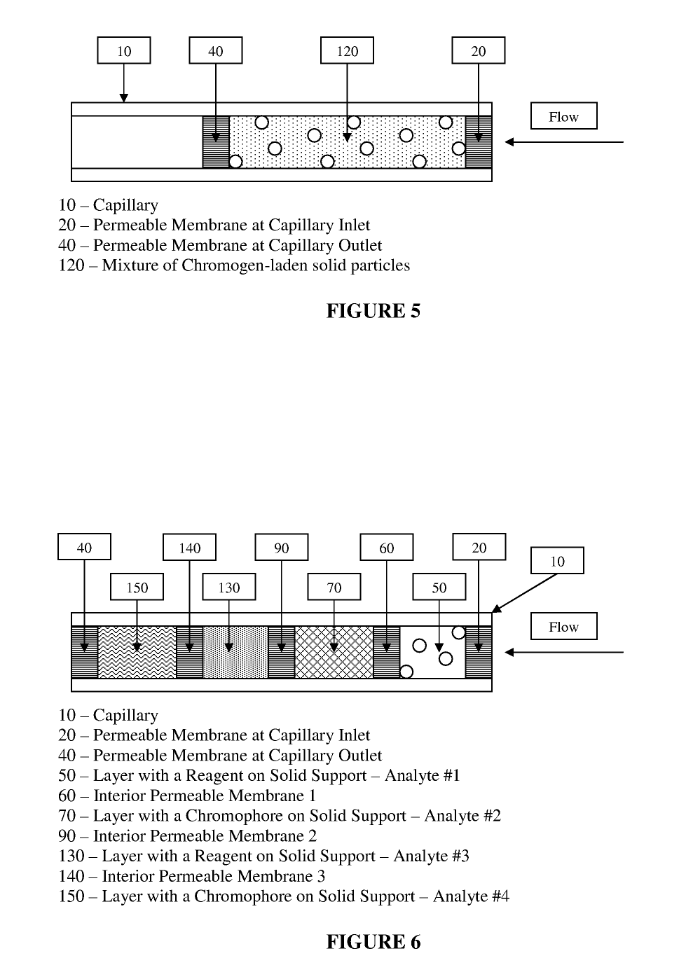

Referring to FIG. 6, the cross-sectional view of another embodiment of the detection capillary is illustrated. In this embodiment multiple layers are used consecutively to allow the simultaneous detection of several analytes using the same capillary. The liquid solution enters through the porous plug 20 and is conditioned in layer 50. The first type of analyte reacts in layer 70 to form one color while the solution continues through the porous plug 90 into a second conditioning layer 130 and then through porous plug 140 into the second chromophoric layer 150 where a second analyte may react to form color. Note that the use of one or both conditioning layers 50 and 130 is not essential in all cases. Moreover, layers 70 and 150 may contain a mixture of the conditioning reagent and the chromophore as described previously.

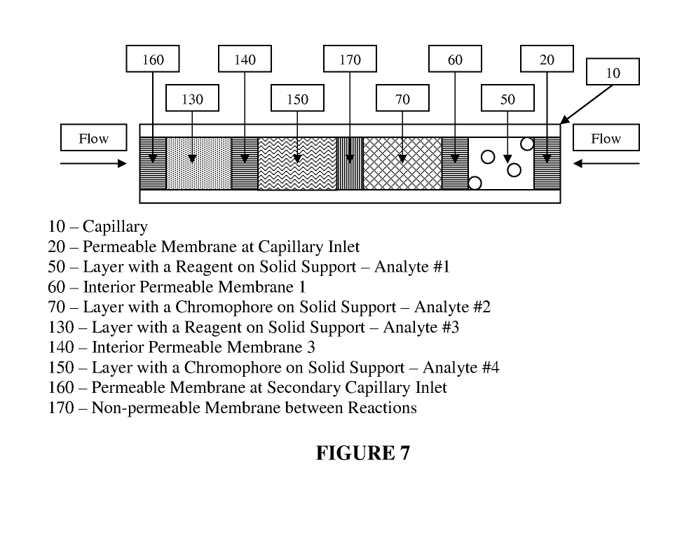

FIG. 7 describes another embodiment of the detector of this invention which allows detecting more than one analyte using the same capillary. The solution is sucked in again by capillary forces through porous plugs 20 and 160 placed on both sides of the capillary. Layers 50 and 130 contain porous solid support with conditioning reagents on it and layers 70 and 150 contain the porous chromophoric solid support. Again, the conditioning layers are not required in all cases. Also, the solid support with the conditioning reagent may be mixed with the chromophoric support to produce a single reactive layer.

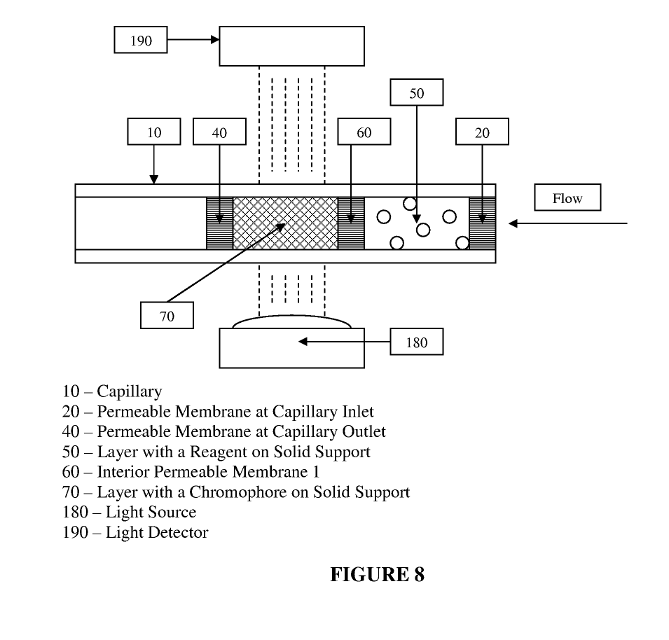

FIG. 8 illustrates schematically one configuration which shows how the capillary detectors can be used in conjunction with a photodetector to obtain quantitative data relative to the concentration of the analyte in the original test solution. A light source 180 is placed on one side of the capillary where the color is expected to be formed and a light detector 190 on its other side. The detector quantifies the modulation of the light by the color formed to yield a quantitative number relative to the analyte concentration. A calibration curve is needed to fully quantify the color.

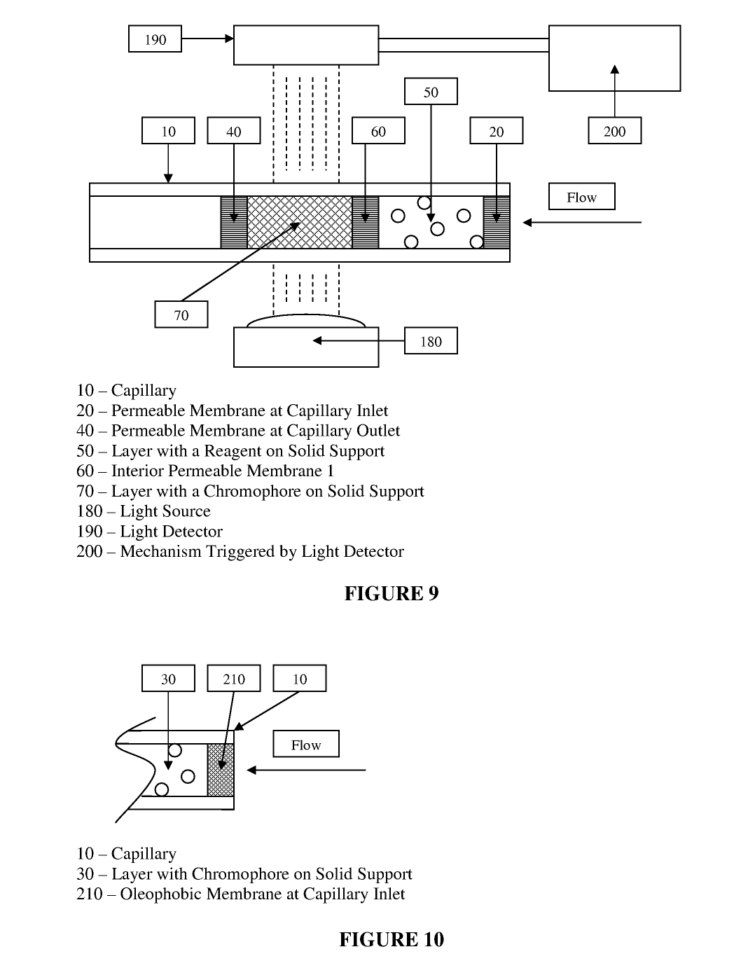

FIG. 9 shows an embodiment of the detector of this invention where the color formed in the capillary between the light source 180 and the light detector 190 is used to trigger the actuation of a secondary equipment 200 such as a pump or a pneumatic valve. These arrangement may be used in cases where a system has to be flashed rapidly as soon as the concentration of a particular analyte reached a critical value.

FIG. 10 shows the entrance section of any of the capillaries. The properties of the porous plug 210 may be tailored to meet specific needs for a particular application. For example, the porous plug 210 may be made out of oleophobic material to deny droplets of oil which may be present in particular liquids from entering the capillary.

As previously introduced, the detection capillary may include written information instructing the user if and/or how much analyte is present, when necessary. Referring to FIG. 4, the analyte concentration may be estimated in a semi-quantitative way by comparing the intensity of the color change to the intensity of a graduated color chart printed on a paper backing the capillary. Importantly, if no color change is observed at the proper location along the capillary, a second capillary detector has to be tested with a solution containing a known and detectable quantity of the analyte(s) to be detected. If color is detected at the proper location along the second capillary, then the validation process is correct and the negative reading is a true negative (and not a false negative).

As previously introduced, the detection capillary may include written information instructing the user if and/or how much analyte is present, when necessary. In cases where various analytes form different colors with the same chromophore, the specific identity of the analyte may presumably be deduced by comparing the color to a color chart placed behind the capillary near the location where the color is expected to be formed. Examples of such capillaries include detection capillary for cyanides, azides and sulfides. The iron-based chromophore used in some of our examples forms blue, red and black colors with cyanides, azides and sulfides, respectively.

In yet another embodiment, the capillaries of the present invention may be sealed following manufacture. The end of the capillary may be notched to facilitate breaking it right before use by a simple bending operation.

The detection capillary is preferably sealed in an envelope that can be readily opened by the user with no tools. For example, the envelope may comprise metallic foil and/or polymeric film (e.g., polyethylene, polypropylene, polyester, etc.), said envelope including marks and/or labels instructing the user on how to open said envelope.

Another embodiment of the present invention is a kit comprising of many detection capillaries for various analytes which may be present in the same sample. The capillaries may be used individually in a sequence or placed in a single holder and dipped simultaneously in the liquid. FIG. 11 illustrates one possible structure which holds multiple capillary detectors.

Another embodiment of the present invention is a kit comprising the detection capillary apparatus and instructions on how to use said apparatus to identify and/or quantify the analyte in a liquid sample. Optional components of said kit may include any or all of the following components as well as other components designed to facilitate the preparation of the sample or the analytical test. These components are a hand-held or small-sized instrumental calorimetric detector, a color chart for identification and/or quantification of the analyte(s), at least one known sample for the quality assurance process, and extraction reagents and instructions relating to the extraction of analyte(s) from some solid samples.

The features and advantages of the present invention are more fully shown by the following non-limiting examples.

### EXAMPLE 1

### Azide and Sulfide Detection Capillary

Chromophore Formulation.

Dissolve 0.25 grams NH4Fe(SO4)2.12H2O in 5 ml water and add to it 3 grams of silica gel with particle size 63-200μ, nominal BET surface area 300 m2/gm. Dry the powder at 150° C. for 30 minutes.

Assembly of Azide Capillary.

1. Take a capillary 100 mm long and 1 mm ID, 1.5 mm OD.

2. Push a small piece of cotton approximately ½ inch into a capillary tube.

3. Fill the capillary to the top with the chromophore.

4. Compact the particles by tapping the capillary 5 times.

5. Plug the inlet of the capillary with another small piece of cotton.

Testing for Azide/Sulfide in Water.

Submerge the opening of the capillary in the water sample for 1-2 seconds and look on the color. Red color indicates the presence of azides and black color indicates the presence of sulfide. The colors form practically instantly.

### EXAMPLE 2

### Cyanide and Chromate Detection Capillary

Chromophore Formulation.

This detection capillary uses two porous support layers, one carrying an activation agent, denoted Mixture A, and the other a chromophore, denoted Mixture B.

Mixture A.

Dissolve 0.1 grams CuSO4 in 5 ml water and add to it 3 grams of silica gel with particle size 63-200μ, nominal BET surface area 300 m2/gm. Dry the powder at 150° C. for 30 minutes.

Mixture B.

Dissolve 5 milligrams tetra methyl benzidine, (TMB), in 5 ml 91% IPA and add to it 3 grams of silica gel with particle size 63-200μ, nominal BET surface area 300 m2/gm. Dry the powder at 150° C. for 5 minutes.

Assembly of Cyanide/Chromate Capillary.

1. Take a capillary 100 mm long and 1 mm ID, 1.5 mm OD.

2. Push a cotton plug approximately ½ inch into a capillary tube.

3. Pack ¼ of an inch of the open volume with Mixture B.

4. Insert a second cotton plug on top of the silica gel

5. Fill the capillary to the top with Mixture A.

6. Compact the particles by tapping the capillary 5 times.

7. Plug the top of the capillary with a small piece of cotton.

Testing for Cyanide/Chromates in Water.

Dip the opening of the capillary in the water sample for 1-3 seconds and look on the color. Blue color indicates the presence of cyanide and blue-violet color indicates the presence of chromates. The colors form practically instantly.

### EXAMPLE 3

### Peroxides Detection Capillary

Chromophore Formulation.

This detection capillary uses two porous support layers, one carrying an activation agent, denoted Mixture A, and the other a chromophore, denoted Mixture B.

Mixture A.

Dissolve 1 gram Na2CO3 in 5 ml water and add to it 3 grams of silica gel with particle size 63-200μ, nominal BET surface area 300 m2/gm. Dry the powder at 150° C. for 30 minutes.

Mixture B.

Dissolve 0.2 grams manganese sulfate in 5 ml water and add to it 3 grams of silica gel with particle size 63-200μ, nominal BET surface area 300 m2/gm. Dry the powder at 150° C. for 30 minutes.

Assembly of the Peroxides Detection Capillary.

1. Take a capillary 100 mm long and 1 mm ID, 1.5 mm OD.

2. Push a cotton plug approximately ½ inch into a capillary tube.

8. Pack ¼ of an inch of the open volume with Mixture B.

9. Insert a second cotton plug on top of the silica gel

10. Fill the capillary to the top with Mixture A.

11. Compact the particles by tapping the capillary 5 times.

12. Plug the top of the capillary with a small piece of cotton.

Testing for Peroxides in Water.

Dip the opening of the capillary in the water sample for 1-3 seconds and look on the color. Black-Brown color indicates the presence of peroxides. The colors form practically instantly.

### EXAMPLE #4

### Flammables Detection Capillary

Chromophore Formulation.

This detection capillary uses two porous support layers, one carrying an activation agent, denoted Mixture A, and the other a chromophore, denoted Mixture B.

Mixture A.

Dissolve 10 milligram 1-(2-pyrdylazo)-2-naphthol in 5 ml acetone and add to it 3 grams of silica gel with particle size 63-200μ, nominal BET surface area 300 m2/gm. Dry the powder at 150° C. for 5 minutes.

Mixture B.

Dissolve 0.5 grams zinc chloride in 5 ml water and add to it 3 grams of silica gel with particle size 63-200μ, nominal BET surface area 300 m2/gm. Dry the powder at 150° C. for 30 minutes.

Assembly of the Flammables Detection Capillary.

1. Take a capillary 100 mm long and 1 mm ID, 1.5 mm OD.

2. Push a cotton plug approximately ½ inch into a capillary tube.

3. Pack ¼ of an inch of the open volume with Mixture B.

4. Insert a second cotton plug on top of the silica gel

5. Fill the capillary to the top with Mixture A.

6. Compact the particles by tapping the capillary 5 times.

7. Plug the top of the capillary with a small piece of cotton.

Testing for Peroxides in Water.

Dip the opening of the capillary in the water sample for 1-3 seconds and look on the color. Red color indicates the presence of peroxides. The colors form practically instantly. Typically, materials like acetone or iso-propanol will form a large diffused zone of color while hydrocarbons such as hexane or octane will form an intense red line.

### EXAMPLE 5

### Arsenic, Antimony and Germanium Compounds

Chromophore Formulation.

This detection capillary uses three porous support layers, one carrying an activation agent, denoted Mixture A, a reactive layer, denoted Mixture B, and a chromophore, denoted Mixture C.

Mixture A.

Dissolve 2 gram citric acid in 5 ml water and add to it 3 grams of silica gel with particle size 63-200μ, nominal BET surface area 300 m2/gm. Dry the powder at 150° C. for 30 minutes.

Mixture B.

Mix 1 grams of zinc dust <10 microns with 3 grams of silica gel with particle size 63-200μ, nominal BET surface area 300 m2/gm.

Mixture C.

Dissolve 0.2 grams sodium bromide and 0.2 grams mercuric bromide in 5 ml water and add to it 3 grams of silica gel with particle size 63-200μ, nominal BET surface area 300 m2/gm. Dry the powder at 150° C. for 30 minutes.

Assembly of the Arsenic/Antimony/Germanium Detection Capillary.

1. Take a capillary 100 mm long and 1 mm ID, 1.5 mm OD.

2. Push a cotton plug approximately ¾ inch into a capillary tube.

3. Pack ¼ of an inch of the open volume with Mixture C.

4. Insert a second cotton plug on top of the silica gel.

5. Pack ¼ of an inch of the remaining volume with Mixture B.

6. Insert a third cotton plug on top of the silica gel

7. Pack the remaining volume with Mixture A.

8. Compact the particles by tapping the capillary 5 times.

9. Plug the top of the capillary with a small piece of cotton.

Testing for Arsenic/Antimony/Germanium in Water.

Dip the opening of the capillary in the water sample for no more than 1-3 seconds and look on the color. Yellow, brown or black color in the end of the capillary indicates the presence of arsenic, antimony or germanium. The colors form in 12 to 30 seconds.

### EXAMPLE 6

### Semi-Quantitative Analysis of Arsenic, Antimony and Germanium Compounds

The chemicals and assembly of this detector is the same as that described in Example 5 but a strip of paper is attached to the capillary with gradations which show the length of the brown stain formed. The length of this stain is related to the amounts of Arsenic, Antimony or Germanium Compounds in the sample.

While the invention has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

What is claimed is:

1. A detection device for sensing the presence and identity of at least one analyte in a liquid sample, said detection device comprising:
a capillary tube;
one or more porous support layers packed consecutively in the tube; and
porous plugs at the entrance to the tube and optionally between the various layers;
at least one layer within the capillary which can interact with at least one analyte present in a test solution or with reaction products of said analyte to form a measurable detectable phenomenon such as a color change or fluorescence.

2. The detection device of claim 1 wherein the capillary tube is adapted to receive sampling material by action of capillary suction forces.

3. The detection device of claim 1 wherein the capillary tube is adapted to receive sampling material by action of capillary suction forces and vacuum applied by a user or by an automatic electronic reader.

4. The detection device of claim 1, wherein said support layer comprises a material selected from the group consisting of paper, modified paper, blotter paper, polymeric beads, porous membranes, porous polymeric particles, porous fibers, gels or soles of organic or inorganic nature, silica powder, alumina powder, ceramic powders, sintered ceramic powders, zirconium oxide, titanium oxides, iron oxides, zinc oxides, thoria, lanthanum oxide, sintered magnesium oxide, aluminosilicates, calcium aluminosilicates, zeolites or molecular sieves, porous sintered monolithic materials, and combinations thereof.

5. The detection device of claim 1, where the measurable detectable phenomenon is a color change visible through the capillary walls.

6. The detection device of claim 1, where the measurable detectable phenomenon is a fluorescence visible through the capillary walls.

7. The detection device of claim 1, where the capillary is constructed of glass.

8. The detection device of claim 1, where the capillary is constructed of a transparent polymer selected from the group consisting of polyacrylates, polyvinylchloride, polyesters, gelatins, tygon, poly-silicones, polyamides, and polyurethanes.

9. The detection device of claim 1, where the porous plugs at the entrance of the capillary and between layers comprised a material selected from the group consisting of cellulosic materials, cotton, polymeric fibers, polyacrylic materials, polyethylene, polypropylene, polyesters, wool, glass wool, sintered beads of polymers, sintered beads of polyethylene, polypropylene, polyesters, and/or polyurethanes, and sintered beads of ceramic materials.

10. The detection device of claim 1, where the porous support layer past the porous plug at the entrance of the capillary is laden with a material that can interact with the analyte and form a measurable change comprising a color change.

11. The detection device of claim 1, where the porous support layer past the porous plug at the entrance of the capillary comprises a mixture of at least two materials laden with materials that can interact with the analyte and form a measurable change comprising a color change.

12. The detection device of claim 1, where the first porous support layer past the porous plug at the entrance of the capillary contains reagents that interact with the analyte or with other components of the liquid to form a material that moves with the flowing solution through a second porous plug and there reacts with a porous support laden with a material that can interact with the analyte and form a measurable change comprising a color change.

13. The detection device of claim 1, where the first porous support layer past the porous plug at the entrance of the capillary contains reagents that interact with the analyte or with other components of the liquid to form a material that moves with the flowing solution through a second porous plug where it reacts with a second material placed on a porous support to form a material that can move with the solution through a third porous plug to react with a porous support laden with a material that can interact with the analyte and form a measurable change comprising a color change.

14. The detection device of claim 1, where the porous support layer past the porous plug at the entrance of the capillary contains reagents that interact with the analyte or with other components of the liquid to form a material that can move with the flowing solution through a second porous plug and react there with a material to form a gaseous product that migrates through a third porous plug into a layer of porous support laden with a material that can interact with the gas and form a measurable change such as a color change.

15. The detection device of claim 1 where printed material is attached to the capillary to allow the user to compare the color formed with the printed color and obtain information relative to the identity of the analyte detected and/or its concentration.

16. The detection device of claim 1 wherein printed material is attached to the capillary or etching is placed on the capillary to allow the user to estimate the analyte concentration based on the length of a color stain formed.

17. The detection device of claim 1, wherein multiple layers of porous support and porous plugs are placed so that there is more than one layer that is laden with reagents that interacts with more than one analyte to form a different measurable change comprising different color changes with different analytes, to allow the detection of multiple analytes using a single capillary detector.

18. The detection device of claim 1, adapted to allow sample to enter the capillary detector from both ends, with multiple layers of porous support and porous plugs placed therein so that there is more than one layer that is laden with reagents that interact with more than one analyte to form different measurable change comprising different color changes with different analytes, to allow the detection of multiple analytes using a single capillary detector.

19. The detection device of claim 1, wherein the color change is due to a chromophore and comprises at least one compound selected from the group consisting of molybdates, phosphomolybdates, tungstates, phosphotungstates, iron sulfates, zinc sulfides, calcium sulfides, barium sulfides, aluminum sulfides, strontium sulfides, nercuric iodide, mercuric iodide complexes, mercuric bromide, mercuric bromide complexes, selenium sulfide, 8-hydroxy-quinoline and its derivatives, 1-(2-pyidylazo)-2-napthol (PAN), 4-(2-Pyrdylazo)-Resorcinol, (PAR), 1-(2-Thiazo-lylazo)-2-Naphthol, (TAN), 4-(2-Thiazo-lylazo)-resorcinol, (TAR), rubeanic acid, diethyldithiocarbamate, dithizone, zincon, ferron, cadion, thoron, arsenazo I, arsenazo III, diphenylcarbazone, diphenylcarbazide (DPC), rhodizonic acid and its salts, titan yellow, cadion, chromotrope IIB, functionalized arsenic diazonium salts, functionalized phosphonic diazonium salts, triphenylmethane, xanthenes, pH indicators and combinations thereof.

20. The detection device of claim 1 wherein the porous support comprises at least one of silica, activated silica and silica gel particles.

21. The detection device of claim 1 wherein the porous support comprises at least one of alumina, activated alumina and alumina gel particles.

22. The detection device of claim 1 wherein the porous plug comprises at least one of cotton, pulp and glass wool.

23. The detection device of claim 1, wherein the chromophore comprises a mixture of iron sulfates deposited on silica particles.

24. The detection device of claim 1, wherein the chromophore is a pH indicator or a mixture of pH indicators.

25. The detection device of claim 1, wherein the at least one analyte is selected from the group consisting of organophosphonates, arsenic compounds, nitrites, nitrates, sulfates, sulfides, ammonia, amines, alcohols, ketones, aldehydes, carbamates, cyanides, azides, sulphites, chlorides, bromides, iodides, hydrazines, thallium ions, mercury ions, copper ions, cadmium ions, lead ions, iron ions, calcium ions, magnesium ions and practically all metallic ions, actinide salts, lanthanide salts, arsenite salts, arsenate salts, chromate salts, selenium compounds, sulfur mustards, arsenic mustards, and lewisite.

26. The detection device of claim 14, wherein the first layer past the porous plug contains an acid deposited on the porous support and the second layer contains fine metal particles that can react with the acid to form a reducing media that is capable of reducing various compounds or analytes to form a gas selected from the group consisting of arsine, germane, hydrogen sulfide, antimony hydride, and phosphine, which subsequently can be detected using a chromogenic reaction with mercuric compounds to form a yellow, brown or black color.

27. The detection device of claim 12, wherein the first reagent comprises a chromogene that can dissolve in organic solvents or in their solutions in water, but that does not dissolve in water, deposited on alumina or silica, and which, once dissolved, moves with the solution through the second porous plug to react with a reagent deposited on the second layer to form a measurable phenomena comprising a color change or fluorescence.

28. The detection device of claim 12 wherein the chromophore on the first porous layer comprises PAN and the reagent on the second comprises zinc, lead, mercury or cadmium ions that react chromogenically with the PAN in the moving solution to form a red or other color.

29. The detection device of claim 12, wherein the reagent placed on the first layer is adapted to react with the analyte to form a soluble compound that reacts chromogenically with a chromogene placed in the second layer to form a color change.

30. The detection device of claim 28 wherein the reagent in the first porous layer comprises an alkaline salt comprising sodium or potassium carbonate or acetate and the reagent on the second contains manganese ions that react chromogenically with peroxides in alkaline media to form a black color.

31. The detection device of claim 28 wherein the reagent in the first porous layer reacts with the analyte to form a compound that reacts differently than the original analyte and that is carried with the solution through the second porous plug to react with a chromophore to form a visible color change.

32. The detection device of claim 28 where the reagent in the first porous layer comprises a copper compound that is reactive with a cyanide ion to form a compound that moves with the solution and oxidizes a homolog of benzidine selected from the group consisting of tetra methyl benzidine, di-methoxy benzidine, di-methyl-benzidine, and o-toulidine, to form a visible color change commensurate with an original concentration of cyanide in the sample.

33. The detection device of claim 13 wherein the reagent in the first porous layer comprises an acid that dissolves in the solution and moves with it through the second porous plug to react with a metal selected from the group consisting of zinc, iron, magnesium, and aluminum, to form a reducing media that reduces the analyte and makes it amenable to react with a chromogenic reagent deposited on the porous support of a third layer where it forms a visible color change commensurate with the original concentration of analyte in the sample.

34. The detection device of claim 33 where the analyte comprises nitrate ion that is reduced in the acidic media carried by the moving solution from the first layer into the second porous support layer laden with elementary zinc mixed with silica particles, where it forms nitrite ion from the nitrates, which reacts in the third porous layer with a mixture containing at least one aromatic amine and optionally a phenol, an aromatic amine or other activated aromatic compound.

35. The detection device of claim 34 where the aromatic amine is selected from the group consisting of sulfanilic acid, antaranilic acid, naphthyl amines, naphthyl amine sulfonates, naphtyl amine benzoates, amino phenols, amino naphthols and homologs of the foregoing compounds, and ring compounds containing nitrogen, sulfur, and/or oxygen therein.

36. The detection device of claim 1 wherein colloidal gold with antibodies is deposited on the first layer and corresponding receptors are placed on the second layer to detect bio-active materials by their immune properties.

37. The detection device of claim 1 wherein antibodies with peroxidase are deposited in or on the support on the first layer and corresponding receptors and an aromatic amine are placed on the second layer to detect corresponding bio-active materials by their immune properties.

38. The detection device of claim 36 wherein the antibodies are for a bioagent selected from the group consisting of bioagents for mad-cow disease, anthrax, e-coli, and salmonella.

39. The detection device of claim 37 wherein the antibodies are for a bioagent selected from the group consisting of bioagents for mad-cow disease, anthrax, e-coli, and salmonella.

40. The detection device of claim 1, wherein the liquid sample comprises a sample selected from the group consisting of water, liquid food, extracts from solid food, ground water, industrial water, waste water, waste dumps fluids, and chemical processing fluids.

41. The detection device of claim 1, comprising a quality assurance layer or a sequence of layers that can be accessed through a second side of the capillary by a known reference solution to form a color confirming that the detector is working correctly, to thereby assure the quality of the detection.

42. The detection device of claim 1, sealed from one or both sides and openable by breaking the capillary at specific notched areas to allow for liquid to be sucked into the capillary and for gases to vent from the capillary.

43. The detection device of claim 1, further comprising a sealable envelope adapted to increase the shelf life and protect the capillary detector during storage and shipment.

44. The detection device of claim 43, wherein the sealable envelope comprises a metallic foil coated with a polymeric film.
